(19) United States
(12) Patent Application Publication
(10) Pub. No.: US 2012/0277863 A1
(43) Pub. Date: Nov. 1, 2012

(54) Maphbe 1

(76) Inventors: Marc Gregory Allinson, Kingsville, MD (US); Phillip Royce Miller, Joppa, MD (US)

(21) Appl. No.: 13/506,223

(22) Filed: Apr. 5, 2012

Related U.S. Application Data
(60) Provisional application No. 61/516,574, filed on Apr. 5, 2011.

(51) Int. Cl. B32B 37/14; A61F 2/44

(52) U.S. Cl. 623/17.14; 156/61

(57) ABSTRACT

The Maphbe 1: Artificial Tissue and Joint Replacement System is a method of replicating the physiological function of tissue as described in the claims. The Maphbe 1 is unique because it employs methods of manufacturing needed to successfully combine the different materials needed to replicate tissue.

### CROSS REFERENCE

This Invention, The Maphbe 1, claims the filing date of provisional patent 61/516,574 with priority filing date Apr. 5, 2011.

### TECHNICAL FIELD

The present invention relates to methods of constructing artificial joints and tissue for the human or animal body.

### BACKGROUND

Many people suffer from degenerative joints, and many have tried to replicate the physiological function of human joints, however they have been unsuccessful. There is a systemic problem with existing orthopedic implants; existing technology does not successfully replicate soft tissue, and furthermore, existing total joint replacements do not utilize a physiologically accurate system for replicating soft tissue in conjunction with metal on metal, metal on polymer, polymer on polymer joints. The absence of soft tissue leads to a systemic problem of fixating the joint in place, furthermore these joints release micro particles into the body when they wear. The Maphbe 1 is designed and tested to solve these problems. The Maphbe 1 was invented by two friends to fix ones spine.

### BRIEF SUMMARY OF THE INVENTION

The Maphbe 1 is a method of replicating the physiological function of tissue

### DETAILED DESCRIPTION OF THE INVENTION

The Maphbe 1 Artificial Tissue and Joint System, referred to as the maphbe 1 or artificial tissue, is a method of replicating the physiological function of tissue as described in the claims. The Maphbe 1 is unique because it employs methods of manufacturing needed to successfully combine the different materials. The Maphbe 1 bonds woven low friction inelastic fiber braids in a contracted/relaxed state with elastomer, such that the elastomer's stretch is limited and the weave returns to its original shape when stress is applied. Normally, low friction fibers do not bond well with elastomer; the Maphbe 1 solves this problem by first interweaving low friction fibers with fibers capable of bonding with elastomer, including but not limited to silk fibers. By combining fibers with different bonding capabilities, the elastomer and fibers can be successfully integrated together. This method can be applied to natural and synthetic elastomers and fibers as well as integrated into tissue and can utilize elatromagnetic structures. Furthermore, the maphbe 1 can utilize heat curing elastomer while maintaining the structural integrity of the fibers by first coating the fibers in an insulative coating before applying heat curing elastomer. This artificial tissue can be used to make ligaments, retinaculum, muscles, breast implants, tendons, joints, patches, membranes, discs, cartilage, meniscus, labrums, as well as be used to coat artificial joints to contain microparticals and fixate.

1) A method of replicating human or animal tissue by interweaving inelastic and/or elastic fibers and then infusing and bonding the weave in a relaxed and/or contracted state with an elastomer including but not limited to air or heat curable elastomer, natural elastomer, organic elastomer, inorganic elastomer, hydrophilic elastomer biological elastomer, natural tissue such that the stretch of the elastomer is limited by the inelastic fibers and such that the elastomer/tissue and/or elcromagnetic structures causes the inelastic fiber weave to return to its original relaxed state after being stretched and contracted, amount stretch/contraction alterable, with or without the aid of electromagnetic structures, stem cells, valves, screws, pulley, ties, coatings, structures, clamps, fusing structures, joints, hooks, loops, glue, bone growth factor and agents, nerve growth factor, collagen growth factor, bmp, allographs, homographs, attachment structures and methods, artificial joint structures, cages, sheaths, mechanisms, meshes, braids, monofilaments, multifilament, plates, wavelength sensitive structures ; hereby referred to as artificial tissue.

2) Pursuant to claim 1, A method of maintaining the structural integrity of woven fibers including but not limited to ultra-high molecular weight polyethylene, polypropylene, nylon, and/or other fibers when infused with a heat curing elastomer by first coating the fibers in a heat insulating coating including but not limited to air curing silicone before infusing with heat curing elastomeric.

3) Pursuant to claim 1, A method of interweaving and/or coating ultra-high molecular weight polyethylene, polypropylene, thermoplastic, nylon, electro-conduct fibers, and/or other low friction fibers and/or low friction carbon tubules, with fibers capable of bonding with silicone elastomeric, natural tissue, collagen, hydrophilic elastomer, light curing elastomer, air curing elastomer, heat curing elastomer, chemical curing, natural elastomer, biological elastomber, inorganic elastomer, organic elastomer, thermoplastic elastomer, and/or other elastomer, bondable fibers including but not limited to silk, such that the low friction fibers and the bonding capable fibers are woven together into a bondable filament that shares the properties of the various fibers of its composition, filaments can be bonded before being transformed into more structures, the bondable filament is woven into structures and bonded with elastomer; the structure maintaining the strength and function of the various fibers, filaments, and elastomers of its compositon, such that the stress, bending, stretch, contraction, and or compressible is alterable and limitable.

4) Pursuant to claim 1, a method, of fixating/coating an artificial “ball and socket” artificial joint using artificial tissue whether elastic and/or inelastic; including but not limited to hips and shoulders.

5) Pursuant to claim 1, the artificial tissue can be used to replicate retinaculum.

6) Pursuant to claim 1, the artificial tissue can be used to replicate all types of muscles.

7) Pursuant to claim 1, the artificial tissue can be used as a shell for breast implants.

8) Pursuant to claim 1, the artificial tissue can be used to make ligaments.

9) Pursuant to claim 1, the artificial tissue can be used to make labrums.

10) Pursuant to claim 1, inelastic fibers can be coated in silicone pressure sensitive adhesive

11) Pursuant to claim 1, the inelastic fibers can be porous and/ or rough surfaced for elastomeric bonding and tissue in growth purposes.

12) Pursuant to claim 1, the artificial connective tissue, polymer, and/or viscoelastic material can be used to contain micro particles released into the body from the wear of orthopedic implants.

13) Pursuant to claim 1, the artificial connective tissue can be woven into sheets for hernia patches and labrums.

14) Pursuant to claim 1, the artificial connective tissue can be woven into cushions for spinal discs replacements, meniscus, and cartilage.

15) Pursuant to claim 1, the artificial connective tissue and can be porous to allow natural tissue in growth,

16) Pursuant to claim 1, the artificial connective tissue can be coated and/or infused with elastomeric, polymers, collagen growth factor, stem cells, allograph ligaments, homograph ligaments, low friction coating, chondroiten sulfate, and/or embroidered microfibers.

17) Pursuant to claim 1, the artificial connective tissue can be used to fixate/coat artificial knee, and elbow joint replacements

18) Pursuant to claim 1, the artificial connective tissue can attach and incporated into tissue, it can be attached metal to metal, bone to bone, bone to metal, polymer to polymer, bone to polymer, and polymer to metal, through any attachment method.

19) Pursuant to claim 1, the artificial tissue can be coated in anti-pathogenic materials, and anticoagulation materials.

20) An artificial spinal joint replacement to be used for cervical, lumbar, and/or thoracic spinal joint replacement; composed of two endplates, a ball and socket joint, coated/fixated/held together/in place by artificial connective tissue, elastic and/or inelastic fibers, elastomeric, polymers; the position, dimensions, and composition of the ball and socket can change and the composition of the endplates can change including but not limited to ring blades, keels, screws, rough titanium etc; as can the composition of the coating and the method of connecting the coating to the endplates; to be used to treat degenerative disc disease and joint instability/spondylolysthesis/spondylolysis.

21) Pursuant to claim 18, a cushion can be integrated into the spinal joint to create a cushioned, fixated, ball and socket spinal joint replacement to be used to treat degenerative disc disease and joint degeneration.

22) Pursuant to claim 18, the spinal joint can be implanted interior, posterior, lateral, bi-lateral, and/or diagonal.
